(19) United States
(12) Patent Application Publication
(10) Pub. No.: US 2026/0234555 A1
(43) Pub. Date: Aug. 13, 2026

(54) METHOD FOR NEUROLOGICAL SIALADENOSIS MODELING AND DRUG EFFICACY TESTING THROUGH CO-CULTURE OF HUMAN SALIVARY GLAND ORGANOIDS AND NEUROCYTOMAS

(71) Applicant: UIF (University Industry Foundation), Yonsei University, Seodaemun-gu, Seoul (KR)

(72) Inventors: Jae-Yol Lim, Gangnam-gu, Seoul (KR); Yeo-Jun Yoon, Seodaemun-gu, Seoul (KR)

(21) Appl. No.: 19/131,190

(22) PCT Filed: Nov. 20, 2023
(86) PCT No.: PCT/KR2023/018698

§ 371 Date: May 19, 2025

(30) Foreign Application Priority Data
Nov. 23, 2022 (KR) 10-2022-0158271

(51) Int. Cl. C12N 5/071; G01N 33/50

(52) U.S. Cl. CPC C12N 5/0633; G01N 33/5082; C12N 2501/113; C12N 2501/115; C12N 2501/117; C12N 2501/119; C12N 2501/15; C12N 2501/155; C12N 2501/415; C12N 2501/727; C12N 2502/30; G01N 2333/33; G01N 2500/00

(57) ABSTRACT

The present disclosure relates to a co-culture of salivary gland organoids and neuroblastoma, and a preparation method therefor and use thereof.

In the present disclosure, organoids based on epithelial cells derived from human salivary gland tissues are cultured and are co-cultured together with neuroblastoma so that a cell-cell direct interaction environment is constructed, and thus a neurological sialadenosis model is provided. In addition, the neurological sialadenosis model is used to screen for novel therapeutic agents and identify responses to a botulinum toxin drug, and thus the present disclosure can contribute to providing patient-customized treatment.

### TECHNICAL FIELD

The present disclosure relates to a co-culture of salivary gland organoids and neuroblastoma, and a preparation method thereof, and use thereof.

### BACKGROUND ART

Sialosis (also known as sialadenosis) is a non-inflammatory, non-neoplastic, parenchymal lesion clinically characterized by recurrent bilateral salivary gland swelling, which is bilateral and symmetrical. It is often painless, but in some instances, tenderness may be present. The known causes of sialadenosis are known to include nutritional deficiencies, various types of endocrine disorders, or autonomic nervous system disorders, but neurological sialadenosis also exists, caused by nervous system disorders or hyperactivity due to unknown or uncontrollable stress.

In general, botulinum toxin drugs are used to treat the neurological sialadenosis.

Botulinum toxin is a neurotoxic protein produced by the bacterium Clostridium botulinum. It functionally inhibits the secretion of acetylcholine, which is a neurotransmitter, at the presynapse of the neuromuscular junction, causing flaccid paralysis of the muscles. Recently, the paralytic mechanism of botulinum toxin has been utilized as a therapeutic approach in a variety of system neurological dysfunctions and autonomic nervous indications. However, while botulinum toxin has therapeutic effects on various diseases, it is highly toxic and can be fatal even in very small amounts, so precise control of its concentration is essential when administered to living organisms.

Currently, the LD 50 measurement method through intraperitoneal injection into mice and the CBPA method using a neuroblastoma cell line are widely used to confirm the potency of botulinum toxin, and other methods such as ELISA are also being developed. However, the existing mouse-based method is expected to be banned in the near future due to ethical issues, and is already banned in some countries. In addition, the CBPA method is protected by a patent by a multinational pharmaceutical company, which limits cost competitiveness due to payment of royalties, etc.

Therefore, the present inventor developed a neurological sialadenosis model based on human salivary gland organoids, confirmed its applicability for screening novel therapeutic agents and measuring responses to a botulinum toxin drug, thereby completing the present disclosure.

### DISCLOSURE

### Technical Problem

An object of the present disclosure is to provide a co-culture of salivary gland organoids and neuroblastoma and a preparation method thereof.

Another object of the present disclosure is to provide a neurological sialadenosis model comprising a co-culture of salivary gland organoids and neuroblastoma, and a preparation method thereof.

Still another object of the present disclosure is to provide a method for screening a therapeutic agent for neurological sialadenosis.

Still another object of the present disclosure is to provide a method for identifying responses to a botulinum toxin drug in a neurological sialadenosis model.

### Technical Solution

In the following specification, description of overlapping content will be omitted to prevent any potential confusion arising from redundancy. In other words, the content of the invention is not limited to the following content; rather, it should be construed in accordance with the comprehensive content of the invention.

Hereinafter, the present disclosure will be described in more detail.

In one general aspect, there is provided a method for preparing a co-culture of a salivary gland organoid and a neuroblastoma.

The method for preparing a co-culture of a salivary gland organoid and a neuroblastoma is performed through the following steps:
(a) culturing a salivary gland organoid from a salivary gland tissue-derived epithelial cell in a culture medium containing a transforming growth factor-beta inhibitor (TGF-β inhibitor), a Wnt activator, a bone morphogenetic protein (BMP) inhibitor, a fibroblast growth factor (FGF) family, a receptor tyrosine kinase ligand, and a Rho-associated protein kinase (ROCK) inhibitor;
(b) culturing a neuroblastoma; and
(c) culturing a salivary gland organoid from a salivary gland tissue-derived epithelial cell in a culture medium containing a TGF-β inhibitor, a Wnt activator, a BMP inhibitor, an FGF family, a receptor tyrosine kinase ligand, a ROCK inhibitor, and a cyclic adenosine monophosphate (CAMP) activator.

As used herein, the term “co-culture” means the result of co-culturing cells of the same or different species maintained under conditions suitable for mutual proliferation, and is a collective term that encompasses co-cultured cells, extracellular substances used in the co-culture, and substances produced during the co-culture.

As used herein, “salivary gland” means an organ that produces and secretes saliva. The salivary glands are classified into major salivary glands such as parotid gland, submaxillary gland, and sublingual gland, and minor salivary glands such as mucous gland, which exists in the mucous membrane of the oral cavity and is distributed to various parts of the oral mucosa.

The term “organoid” as used herein refers to a cell aggregate created by coagulating and recombining cells separated from stem cells or organ-derived cells through re-culturing, and may include an organoid or cell cluster formed from a suspension cell culture.

As used herein, the term “neuroblastoma” is a tumor of the nervous system that originates from neural tissue. When it occurs in the central nervous system, it is known to occur mainly in the lateral ventricle of the brain and cause symptoms such as headache and slowed reaction time due to increased intracranial pressure.

The culture medium used in Step (a) of the method for preparing the co-culture of the salivary gland organoid and the neuroblastoma according to the present disclosure is provided for culturing the salivary gland organoid from the salivary gland tissue-derived epithelial cell, and essentially comprises a TGF-β inhibitor, a Wnt activator, a BMP inhibitor, an FGF family, a receptor tyrosine kinase ligand, and a ROCK inhibitor.

The term “culture medium” as used herein means solution for maintaining a cell population or culturing a cell population, which contains nutrients that maintain cell viability and support proliferation.

The culture medium used in the present disclosure comprises a basal medium. The basal medium is any basal medium suitable for the culture of animal or human cells.

The basic medium typically contains a number of components necessary to support the maintenance of cultured cells. Combinations of suitable components may be easily formulated by a skilled person in consideration of the following description. Also included is a nutrient solution containing common standard cell culture components, such as amino acids, vitamins, lipid supplements, mineral salts, carbon energy sources, and buffers.

The basal media are commercially available and include, but are not limited to, Dulbecco's Modified Eagles Media (DMEM), Minimum Essential Media (MEM), KnockOut-DMEM (KO-DMEM), Glasgow's Minimum Essential Media (G-MEM), Eagle's Minimum Essential Medium (EMEM), Basal Medium Eagle (BME), DMEM/Ham's F12, Advanced DMEM/Ham's F12, Iscove's Modified Dulbecco's Media, and Minimum Essential Media (MEM), Ham's F-10, Ham's F-12, medium 199, RPMI 1640 medium, and KnockOut Serum replacement XenoFree medium. For example, the basal medium may be Advanced DMEM/F12 medium.

The TGF-β inhibitor may be any substance that inhibits the function of the TGF-β receptor, for example, a protein, a peptide, a small molecule, and may be any one selected from the group consisting of A83-01, SB-431542, SB-505124, SB-525334, SD-208, LY-36494, and SJN-2511. Preferably, the TGF-β inhibitor may be A83-01. More preferably, the TGF-β inhibitor may be 0.5 μM to 15 μM A83-01. More preferably, the TGF-β inhibitor may be 0.5 μM to 5 μM A83-01.

The Wnt activator may be any one selected from the group consisting of R-spondin 1, R-spondin 2, R-spondin 3, and R-spondin 4. Preferably, the Wnt activator may be R-spondin 3.

The BMP inhibitor may be an agent that binds to a BMP molecule to form a complex, i.e., an agent that binds to a BMP receptor and prevents binding of the BMP ligand to the receptor, such as an antibody that binds to the receptor. The BMP inhibitor may be a protein or a small molecule and may be natural, modified, and/or partially or entirely synthetic. In addition, the BMP inhibitor may be any one selected from the group consisting of Noggin, Dorsomorphin, DMH1, and LDN-1931. Prefereably, the BMP inhibitor may be Noggin.

The fibroblast growth factor (FGF) family is a powerful factor that regulates cell proliferation and differentiation, and plays an essential role in the normal development of stem cells, maintenance of tissue homeostasis, wound healing, and angiogenesis. The FGF family may be at least any one selected from the group consisting of FGF1, FGF2, FGF3, FGF4, FGF5, FGF6, FGF7, FGF8, FGF9 and FGF10. Preferably, the FGF family may be FGF2 and FGF10. More preferably, the FGF family may be 5 ng/ml to 20 ng/ML FGF2 and/or FGF10. More preferably, the FGF family may be 5 ng/ml to 10 ng/ml FGF2 and/or FGF10.

The receptor tyrosine kinase ligand may be any one selected from the group consisting of Neuregulin β1 (NRG1), Heregulin β1 (HRG1), an epidermal growth factor (EGF), a transforming growth factor-α (TGF-α), a basal fibroblast growth factor (bFGF), a brain-derived neurotrophic factor (BDNF), a hepatocyte growth factor (HGF), and a keratinocyte growth factor (KGF). Preferably, the receptor tyrosine kinase ligand may be Neuregulin β1 (NRG1). More preferably, the receptor tyrosine kinase ligand may be 5 ng/ml to 20 ng/mL Neuregulin β1 (NRG1). More preferably, the receptor tyrosine kinase ligand may be 5 ng/mL to 10 ng/mL Neuregulin β1 (NRG1).

The Rho-associated protein kinase (ROCK) inhibitor serves to inhibit the activity of serine/threonine kinase that acts as a target protein for Rho (Rho A, Rho B, and Rho C), and may be any one selected from the group consisting of R-(+)-trans-4-(1-Aminoethyl)-N-(4-pyridyl)cyclohexane carboxamide dihydrochloride monohydrate (Y-27632), Fasudil, and H-1152. Preferably, the ROCK inhibitor may be Y-27632. More preferably, the ROCK inhibitor may be 5 μM to 20 μM Y-27632. More preferably, the ROCK inhibitor may be 5 μM to 15 μM Y-27632.

In addition to the essential components described above, the culture medium of the present disclosure may further comprise any one or more additional components selected from the group consisting of Glutamax, HEPES, Primocin, Prostaglandin E2 (PGE2), N-acetylcysteine (NAC), B27, and Nicotinamide.

The B27 may be replaced with a generic formulation including one or more of the components selected from the following list: biotin, cholesterol, linoleic acid, linolenic acid, progesterone, putrescine, retinyl acetate, sodium selenite, triiodothyronine (T3), DL-alpha-tocopherol (vitamin E), albumin, insulin, and transferrin.

Each of these additional components may be adequately modulated in concentration within the general scope commonly used in culture medium.

More specifically, the culture medium used in Step (a) of the method for preparing the co-culture of the salivary gland organoid and the neuroblastoma according to the present disclosure may comprise A83-01, R-spondin 3, Noggin, FGF2, FGF10, Neuregulin β1 (NRG1), and Y-27632.

The salivary gland tissue-derived epithelial cell according to the present disclosure may be derived from the salivary gland tissue of a subject. The salivary gland tissue may be normal salivary gland tissue or a patient's salivary gland tissue. In addition, the salivary gland tissue may be derived from commercially available salivary gland cells and/or tissues.

The culture medium used in Step (b) of the method for preparing the co-culture of the salivary gland organoid and the neuroblastoma according to the present disclosure may further contain general antibiotics and/or serum components in a culture medium commonly used. For example, the culture medium may contain antibiotics such as P/S or Primocin, serum components such as FBS, etc.

The neuroblastoma may be used by differentiating, for example, commercially available Neuro-2a cells, SK-N-SH cells, SH-SY5Y cells, B35 cells, IMR-32 cells, or NTERA-2 cells, known for their ability to generate teratomas. In addition, one obtained and cultured from a patient's neuroblastoma may be used.

The culture medium used in Step (c) of the method for preparing the co-culture of the salivary gland organoid and the neuroblastoma according to the present disclosure is provided for culturing a mixture of the salivary gland organoid and the neuroblastoma, and essentially comprises a TGF-β inhibitor, a Wnt activator, a BMP inhibitor, an FGF family, a receptor tyrosine kinase ligand, a ROCK inhibitor, and a CAMP activator.

The TGF-inhibitor, Wnt activator, BMP inhibitor, FGF family, receptor tyrosine kinase ligand, and ROCK inhibitor are the same as described above.

The CAMP (cyclic adenosine monophosphate) activator may be any one selected from the group consisting of Forskolin, Aminophylline, Pentoxifylline, Theophylline, Isobutyl-methylxanthine (IBMX), and Dehydroabietic acid (DAA). Preferably, the CAMP activator may be Forskolin. More preferably, the CAMP activator may be 5 μM to 15 μM Forskolin.

More specifically, the culture medium used in Step (c) of the method for preparing the co-culture of the salivary gland organoid and the neuroblastoma according to the present disclosure may comprise A83-01, R-spondin 3, Noggin, FGF2, FGF10, Neuregulin β1 (NRG1), Y-27632, and Forskolin.

The present disclosure provides a medium composition for culturing a co-culture of a salivary gland organoid and a neuroblastoma, comprising: a TGF-β inhibitor, a Wnt activator, a BMP an inhibitor, FGF family, a receptor tyrosine kinase ligand, ROCK inhibitor, and a CAMP activator.

More specifically, the medium composition for culturing a co-culture may essentially comprise A83-01, R-spondin 3, Noggin, FGF2, FGF10, Neuregulin β1 (NRG1), and Y-27632.

In another general aspect, there is provided a co-culture of a salivary gland organoid and a neuroblastoma, prepared according to any one of the methods as described above.

In still another general aspect, there is provided a method for preparing a neurological sialadenosis model.

The method for preparing a neurological sialadenosis model is performed through the following steps:
(a) culturing a salivary gland organoid from a salivary gland tissue-derived epithelial cell in a culture medium containing a transforming growth factor-beta inhibitor (TGF-β inhibitor), a Wnt activator, a bone morphogenetic protein (BMP) inhibitor, a fibroblast growth factor (FGF) family, a receptor tyrosine kinase ligand, and a Rho-associated protein kinase (ROCK) inhibitor;
(b) culturing a neuroblastoma;
(c) preparing a co-culture by mixing and culturing the salivary gland organoid and the neuroblastoma in a culture medium containing a TGF-β inhibitor, a Wnt activator, a BMP inhibitor, an FGF family, a receptor tyrosine kinase ligand, a ROCK inhibitor, and a cyclic adenosine monophosphate (CAMP) activator; and
(d) treating the co-culture with a neurotransmitter.

As used herein, the term “sialosis/sialadenosis” is a non-inflammatory, non-neoplastic, parenchymal lesion clinically characterized by recurrent bilateral salivary gland swelling, which mainly appears in the parotid glands, but in rare cases, in the submaxillary glands and sublingual glands. The swelling is bilateral and symmetrical, and it is often painless, but in some instances, tenderness may be present. The causes of sialosis/sialadenosis are known to include nutritional deficiencies, various types of endocrine disorders, or autonomic nervous system disorders, etc.

The term “neurological sialadenosis” as used herein refers to sialadenosis caused by nervous system abnormality or hyperactivity, such as stress of unknown cause or uncontrollable stress.

The term “neurotransmitter” means a signal substance secreted from a nerve cell, which travels across a synapse and functions to either increase or decrease the membrane potential of an adjacent nerve cell. The neurotransmitter may be any one selected from the group consisting of acetylcholine, dopamine, norepinephrine, serotonin, and histamine. Preferably, the neurotransmitter may be acetylcholine.

According to an embodiment of the present disclosure, the co-culture prepared according to Steps (a) to (c) may be treated with acetylcholine, a neurotransmitter, and/or neostigmine, an inhibitor of its degradation. Here, neostigmine increases the amount of acetylcholine in the neuroblastoma and acetylcholine stimulates the parasympathetic nerves to expand the lumen of the salivary gland organoid, thereby preparing a neurological sialadenosis model.

Preferably, the method according to the present disclosure may comprise treating the co-culture with any one selected from the group consisting of acetylcholine, dopamine, norepinephrine, serotonin, and histamine; and neostigmine.

In another general aspect, there is provided a medium composition for culturing a co-culture of a salivary gland organoid and a neuroblastoma.

The medium composition may comprise a TGF-β inhibitor, a Wnt activator, a BMP inhibitor, an FGF family, a receptor tyrosine kinase ligand, a ROCK inhibitor, and a CAMP activator, preferably A83-01, R-spondin 3, Noggin, FGF2, FGF10, Neuregulin β1 (NRG1), and Y-27632.

In another general aspect, there is provided a neurological sialadenosis model prepared according to the preparation method as described above.

In still another general aspect, there is provided a method for screening a therapeutic agent for neurological sialadenosis.

Specifically, the present disclosure provides a method for screening a therapeutic agent for neurological sialadenosis, comprising: treating a test substance with the neurological sialadenosis model prepared according to the preparation method as described above.

More specifically, the method for screening for a therapeutic agent for neurological sialadenosis may further comprise selecting the test substance as a therapeutic agent for neurological sialadenosis, when an expression level of at least any one selected from the group consisting of KRT7, KRT19, AchE, and Aqp5 is reduced in the neurological sialadenosis model treated with the test substance compared to the control group not treated with the test substance.

The term “screening” means selecting a substance having a specific target property from a candidate group of various substances using a specific manipulation or evaluation method.

The term “test substance” used when referring to the screening method of the present disclosure means an unknown substance used in screening to examine whether it affects the expression levels of KRT7, KRT19, AchE, and Aqp5. The test substance includes, but is not limited to, chemicals, nucleotides, antisense-RNA, shRNA, miRNA, siRNA (small interference RNA), and natural product extract.

The term “selection” as used in referring to the screening method of the present disclosure refers to selecting a substance that reduces the expression level of any one or more specific selection markers, for example, selected from the group consisting of KRT7, KRT19, AchE, and Aqp5.

The term “KRT7 (Keratin 7)” is a member of the keratin gene family corresponding to type II cytokeratin, and type II cytokeratin is composed of a basic neutral protein arranged in pairs of heterotypic keratin chains that are co-expressed during differentiation of monolayer and mesenchymal epithelial tissues. These type II cytokeratins are specifically expressed in the luminal monolayer epithelial layers of internal organs, as well as in ducts and blood vessels.

The term “KRT19 (Keratin 19)” is a member of the keratin gene family and corresponds to type I cytokeratin, and is specifically known to be expressed in the outer epidermis, which serves as a transient superficial layer covering the epidermis during development.

As used herein, the term “AchE (acetylcholinesterase)” plays a role in terminating neurotransmission at synapses by degrading the neurotransmitter, acetylcholine, into acetate and choline, and is also known to be involved in various responses to stress.

The term “Aqp5 (Aquaporin 5)” as used herein is one of the membrane protein families related to major intrinsic proteins and is a factor that plays a crucial role in the production of saliva, tears, and pulmonary secretions.

The present disclosure provides a method for confirming responses to a botulinum toxin drug in a neurological sialadenosis model.

Specifically, the present disclosure provides a method for confirming responses to a botulinum toxin drug in a neurological sialadenosis model, the method comprising: treating the neurological sialadenosis model prepared according to the preparation method with a botulinum toxin drug.

More specifically, the method for confirming the responses to a botulinum toxin drug in a neurological sialadenosis model may further comprise comparing an expression level of any one or more selected from the group consisting of KRT7, KRT19, AchE, and Aqp5 in the neurological sialadenosis model treated with the botulinum toxin drug with that of a control group not treated with the botulinum toxin drug.

The term “botulinum toxin” is a toxin secreted by anaerobic bacteria such as Clostridium botulinum, C. butyricum, C. baratii, and C. argentinense, comprising seven types (A-G) in total, among which botulinum toxin types A and B are purified and used for medical applications. The toxin inhibits the secretion of acetylcholine at the motor nerve terminal, thereby paralyzing the corresponding muscles.

The botulinum toxin drug may be Botox®, Meditoxin, Dysport®, Botulax, Myobloc®, BTX or Xeomin®. Preferably, it may be Botox®. More preferably, it may be Botox® at 1 U/mL to 200 U/mL.

According to an embodiment of the present disclosure, a botulinum toxin receptor, such as GT1b, may be treated in the neurological sialadenosis model, together with the botulinum toxin drug.

The salivary gland tissue-derived epithelial cell according to the present disclosure may be derived from the salivary gland tissue of a subject. The salivary gland tissue may be normal salivary gland tissue or a patient's salivary gland tissue.

According to an embodiment of the present disclosure, the expression levels of lumen-related genes (KRT7 and KRT19), AchE, and Aqp5 of the neurological sialadenosis model may be significantly reduced by treatment with the botulinum toxin drug.

### Advantageous Effects

The present disclosure provides a neurological sialadenosis model by culturing organoids based on epithelial cells derived from human salivary gland tissues and co-culturing the organoids with neuroblastoma so that a cell-cell direct interaction environment is constructed. In addition, the neurological sialadenosis model is used to screen for novel therapeutic agents and identify responses to a botulinum toxin drug, and thus the present disclosure can contribute to providing patient-customized treatment.

### DESCRIPTION OF DRAWINGS

FIG. 1 shows the high culture time and initial cell number of ChAT gene expression, which contributes acetylcholine production for salivary gland organoids, confirmed through qPCR during the suspension culture of N2a cells alone.

FIG. 2 shows the presence or absence of lumen swelling depending on the co-culture of human salivary gland organoids and N2a cells, confirmed through bright-field observation.

FIG. 3 shows immunofluorescence staining for factors expressed in salivary gland organoids co-cultured with N₂a cells and salivary gland organoids that were not co-cultured.

FIG. 4 shows a group treated with neurotransmitters (Acetylcholine+Neostigmine) and a group treated with Botox to block neurotransmitters (GT1b+BONT/A), observed under a bright field microscope.

FIG. 5 shows the expression levels of lumen-related genes (KRT7 and KRT19) in the group treated with neurotransmitters (Acetylcholine+Neostigmine) and the group treated with Botox to block neurotransmitters (GT1b+BONT/A).

FIG. 6 shows a schematic diagram of an experimental schedule to check the effect of Botox on salivary gland organoids.

FIG. 7 shows the changes observed in mouse salivary gland organoids when treated with various concentrations of acetylcholine.

FIG. 8 shows the changes observed in mouse salivary gland organoids when treated with various concentrations of neostigmine.

FIG. 9 shows the changes observed in mouse salivary gland organoids when treated with 25 μg/mL of GT1b and various concentrations of Botox.

FIG. 10 shows a schematic diagram of an experimental schedule for preparing and validating a co-culture system of mouse salivary gland organoids and N2a cells.

FIG. 11 shows the co-culture system of mouse salivary gland organoids and N2a cells observed under a bright-field microscope.

FIG. 12 shows the results of single-cell transcriptome analysis on the co-culture system of mouse salivary gland organoids and N2a cells.

FIG. 13 shows immunofluorescence staining for the factors expressed in the co-culture system of mouse salivary gland organoids and N2a cells.

FIG. 14 shows the expression levels of ChAT genes and AchE genes for the Untreated (−Stim-Botox), Stimulated (+Stim-Botox), and Stimulated+Botox (+Stim+Botox) groups.

FIG. 15 shows the expression levels of Krt5, Krt7, Aqp5, and Bhlha15 for the Untreated (−Stim-Botox), Stimulated (+Stim-Botox), and Stimulated+Botox (+Stim+Botox) groups.

### BEST MODE

The following Experimental Examples are intended to provide Experimental Examples commonly applied to each of Examples according to the present disclosure.

### Experimental Example 1. Reagents Used in the Present Disclosure

Advanced DMEM/F12 (#12634010, Gibco), HEPES (#15630-080, Gibco), GlutaMAX (#35050-061, Gibco), Penicillin/Streptomycin (#15140122, Gibco), Primocin (#ant-pm-2, Invivogen), B-27 (50X) (#12587-010, Gibco), N-acetyl-cysteine (#A9165, Sigma), A83-01 (#2939, Tocris), Y-27632 dihydrochloride (#1254, Tocris), Recombinant Murine R-Spondin-1 (#315-32, Peprotech), Recombinant Human Heregulin beta-1 (Neuregulin-1; #100-03, Peprotech), Recombinant Murine FGF-acidic (#450-33A, Peprotech), Recombinant Murine KGF (#450-60, Peprotech), Collagenase, type II (#4176, Worthington), Matrigel (#356231, Corning), Cell Recovery Solution (#354253, Corning), TrypLE express (#12605-010, Gibco), CellBanker 1 (Zenoaq), 24 well plate for suspension culture (#677102, Greiner Bio-one), 48 well plate for suspension culture (#677102, Greiner Bio-one), 25 cm² cell culture plate (#70025, SPL), Trypan Blue Solution, 0.4% (#15250-061, Gibco), EMEM (#30-2003, ATCC), Fetal bovine serum (#16000-044, Gibco), PBS (#14190144, Gibco), Strainer (#93070, SPL), Falcon 5 mL Round Bottom Polystyrene Test Tube, with Cell Strainer Snap Cap (#352235, Falcon), Protein LoBind Tube 1.5 mL (#022431081, Eppendorf AG), 5 mL conical Tube (#51105, SPL), 15 mL conical Tube (#51115, SPL), 50 mL conical Tube (#51150, SPL), GT1b (#G3767, Sigma), Botulax Inj. 200U (Hugel), TRIzol (#15596018, Thermo Fisher (#RR037A, Takara), SensiFAST Scientific), RT Reagent kit SYBR Lo-ROX kit (#BIO-94020, BIOLINE), Acetylcholine chloride (#A2661, Sigma), Neostigmine bromide (#N2001, Sigma), PGE2 (#2296, Tocris), Forskolin (#1099, Tocris), Recombinant RSPO3-Fc fusion protein conditioned medium (hRSPO3-CM) (R001, U-Protein Express BV), Noggin-Fc Fusion Protein conditioned medium (hNoggin-CM) (#N002, U-Protein Express BV), Recombinant Human FGF-basic (154 a.a.) (#100-18B, Peprotech), Recombinant Human FGF-10 (#100-26, Peprotech), Hematoxylin (#63534, Abcam), Eosin (#64044, Abcam), 100% Ethanol (#1.08543.0250, Merck), Periodic Acid Schiff (PAS) stain kit (#ab150680, Abcam), Permount mounting medium (#SP15-100, Fisherbrand)

### Experimental Example 2. Processing and Storage of Human Salivary Stem Cells

Salivary gland samples were stored at 4° C. in Advanced DMEM/F12 containing 1× Glutamax and 1×HEPES (basal medium). A digestion solution was prepared by adding 5 mg/mL Collagenase type II and 10 μM Y-27632 to Advanced DMEM/F12, calculated as 1 mL per 50 mg of salivary sample, and the salivary gland sample was minced with a blade and transferred to a new conical tube along with the digestion solution.

The reaction mixture was incubated at 200 rpm for 1 hour in a 37° C. shaker and centrifuged at 300 g for 5 minutes, and the supernatant was removed. TrypLE express 2 mL and Y-27632 10 μM were added to the cells with supernatant removed, pipetted, and incubated at 37° C. for 10 minutes.

The salivary gland samples were filtered through a 100 μm strainer to remove undigested tissue, and 2 mL of Advanced DMEM/F12 was added to stop the reaction, followed by centrifugation at 300 g for 5 minutes to obtain a cell pellet. The cell pellet was resuspended in CellBanker 1, calculated to contain at least 2×10⁵ cells per cryovial (1 mL), and 1 mL was dispensed into each cryovial, stored at −80° C. for 2 to 3 days, and then transferred to a nitrogen tank for long-term storage.

### Experimental Example 3. Culturing of Salivary Gland Organoids Derived from Human Salivary Gland Stem Cells

The cell stock was taken out from the nitrogen tank and incubated at 37° C. until half dissolved, then centrifuged at 300 g for 5 minutes to remove the supernatant and obtain a cell pellet. Then, 1 mL of organoid culture medium was added to fully dissolve the cell pellet, a 10 μL aliquot was collected and transferred to a 1.5 mL Eppendorf tube, and 10 UL of Trypan Blue Solution 0.4% was added and mixed. The number of cells (cell count) and viability were checked.

5.0×10³ cells were dispensed per well in a 48-well plate, and 20 μL of Matrigel was added per well and mixed with the cells. 20 μL of the cell and Matrigel mixture was embedded and solidified into a dome shape, and 300 μL of culture medium was added and cultured at 37° C. The composition of the culture medium m employed during this experiment is as shown in Table 1 below.

TABLE 1

| Advanced DMEM/F12 | Basal medium | |
|---|---|---|
| Glutamax | 1 | X |
| HEPES | 1 | X |
| Primocin | 0.2% | |
| B27 vit A+ | 1 | X |
| NAC | 1 | mM |
| Nicotinamide | 5 | mM |
| A83-01 | 5 | μM |
| PGE2 | 3 | μM |
| hRSPO3 CM | 1% | |
| hNoggin CM | 2% | |
| hNRG1 | 5 | ng/mL |
| hFGF2 | 5 | ng/mL |
| hFGF10 | 10 | ng/mL |
| Y-27632 | 10 | μM |

The existing culture medium was replaced with new culture medium every 2 to 3 days, and Y-27632 was removed from the culture medium after the first 3 days. In addition, subculture was performed prior to the completion of 14 days.

### Experimental Example 4. Culture and Subculture (Passage) of N2a Cells

The cell stock (total cell count: at least 5.0×10⁵) was taken out from the nitrogen tank and incubated at 37° C. until half melted, and a neutralization solution containing 10% FBS in EMEM was added, followed by centrifugation at 300 g for 5 minutes to remove the supernatant. Next, the neutralization solution was added again and centrifuged at 300 g for 5 minutes to obtain a cell pellet. After dissolving the cell pellet by adding 1 mL of culture solution, a 10 μL aliquot was collected and transferred to an Eppendorf tube, 10 μL of Trypan Blue Solution 0.4% was added, and the cell count was confirmed.

5.0×10⁵ cells were dispensed into a T25 plate and placed with the culture medium. The composition of the culture medium employed during this experiment is as shown in Table 2 below.

TABLE 2

| EMEM | Basal medium |
|---|---|
| FBS | 10% |
| Primocin | 0.2% |

When the cells reached 80-90% confluency, subculture was performed. The culture medium was removed, and the surface was gently washed with Ca²⁺ Mg²⁺ free PBS. Then, 2 mL of TrypLE was added and incubated at 37° C. The cells were examined under a microscope every 5 minutes until most of the cells became a single-cell suspension. Upon confirmation that the cells were in a single-cell form, the cells were transferred to a conical tube, and the same amount of neutralization solution was added and centrifuged at 300 g for 5 minutes to remove the supernatant and obtain the cell pellet. The cell pellet was dissolved by adding 1 mL of culture solution, and a 10 μL aliquot was collected and transferred to an Eppendorf tube. 10 μL of Trypan Blue Solution 0.4% was added and mixed, then the cell count was checked, and cell culture was performed repeatedly.

The following Examples are presented to enhance comprehension of the present disclosure. These Examples are only provided to more easily understand the present disclosure, and do not impose limitations on the content of the present disclosure.

### Example 1. Suspension Co-Culture System of Human Salivary Gland Organoids and N2a Cells

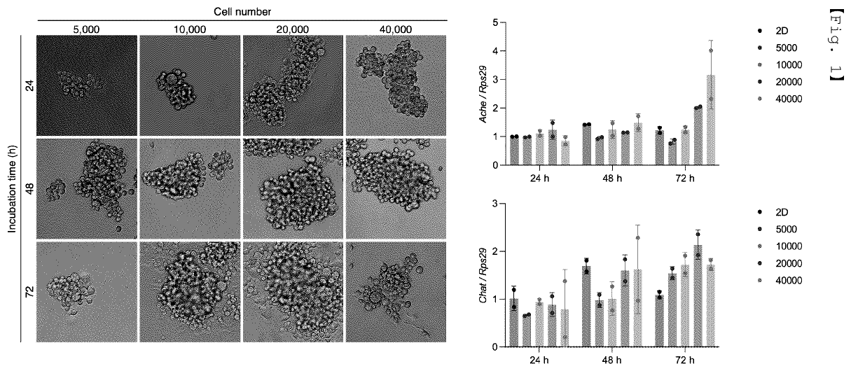

Prior to proceeding with suspension co-culture, the high culture time and initial cell number of ChAT gene expression, which contributes acetylcholine production for salivary gland organoids, confirmed through qPCR during the suspension culture of N2a cells alone, were set to 24 hours and 1.0×10⁴ cells, respectively (FIG. 1). Therefore, the organoids were subjected to subculture without Matrigel in a 48-well plate with 1.0×10⁴ cells/300 μL per well. The composition of the culture medium employed during this experiment is as shown in Table 3 below.

TABLE 3

| Advanced DMEM/F12 | Basal medium | |
|---|---|---|
| Glutamax | 1 | X |
| HEPES | 1 | X |
| Primocin | 0.2% | |
| B27 vit A+ | 1 | X |
| NAC | 1 | mM |
| Nicotinamide | 5 | mM |
| A83-01 | 5 | μM |
| PGE2 | 3 | μM |
| hRSPO3 CM | 1% | |
| hNoggin CM | 2% | |
| hNRG1 | 5 | ng/mL |
| hFGF2 | 5 | ng/mL |
| hFGF10 | 10 | ng/mL |
| Y-27632 | 10 | μM |
| Forskolin | 10 | μM |

After 72 hours, 100 μL of the above culture medium was added, and after another 72 hours, N2a cells were diluted 1.0×10⁵ in 1 mL of the culture medium and 100 μL was added to the wells for co-culture. After 24 hours, the experiment was terminated and post-experimental analysis was performed.

### Example 2. Confirmation of Suspension Co-Culture System of Human Salivary Gland Organoids and N2a Cells

### 2.1. Lumen Swelling

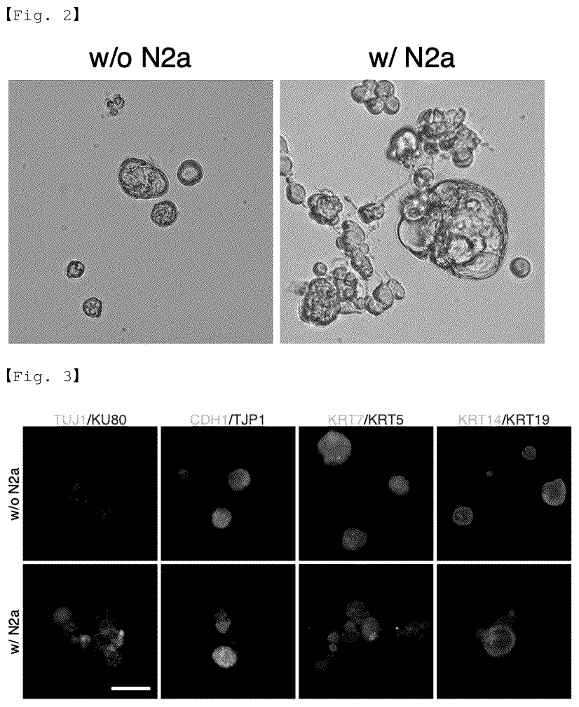

The presence or absence of lumen swelling depending on the co-culture of human salivary gland organoids and N2a cells was compared through bright-field observation (FIG. 2). The results showed that the lumen of the salivary gland organoids was expanded only when co-cultured with N₂a cells.

### 2.2. Immunofluorescence Staining

Immunofluorescence staining was performed to confirm that KU80, which marks the nucleus of human cells, and TUJ1, which is expressed only in N2a cells but not in salivary gland epithelial cells, were expressed in the co-culture system of human salivary gland organoids and N2a cells. Meanwhile, salivary gland epithelial cell markers such as CDH1, KRT7, KRT5, KRT14, and KRT19 were expressed in both the groups co-cultured with N₂a cells and groups not co-cultured (FIG. 3).

### Example 3. Neurological Sialadenosis Model Using Suspension Co-Culture System of Human Salivary Gland Organoids and N2a Cells and Drug Efficacy Test Using the Same

In order to confirm the treatment effect of neurotransmitters such as acetylcholine and neostigmine on the co-culture system, bright field observation was performed on the group treated with neurotransmitters (Acetylcholine+Neostigmine) and the group treated with Botox to block neurotransmitters (GT1b+BONT/A), and the expression levels of lumen-related genes (KRT7 and KRT19) were measured.

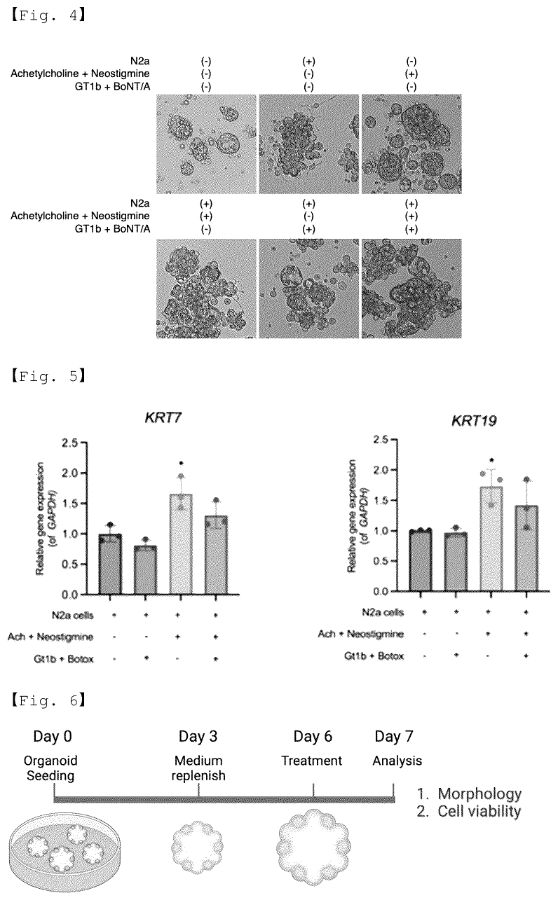

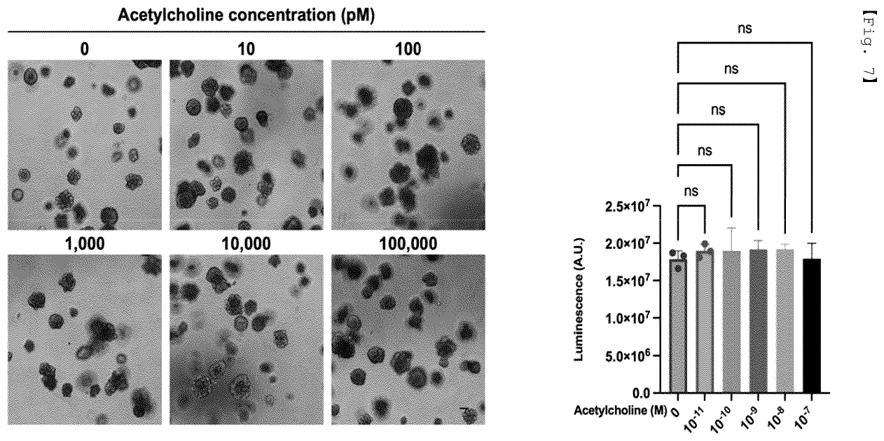

Bright field microscopy confirmed that even though the lumen in the organoid was swollen by the co-culture with N₂a cells and the neurotransmitters, the lumen contracted when the neurotransmitters were blocked (FIG. 4). In addition, the expression of genes related to the lumen was confirmed, and as a result, it was confirmed that the expression levels of KRT7 and KRT19 increased when the neurotransmitters were supplied and decreased when the neurotransmitters were blocked (FIG. 5).

In other words, it was confirmed that the neurological sialadenosis model could be effectively prepared by treating the neurotransmitters in the co-culture system of human salivary gland organoids and N2a cells. This is considered because the treatment with acetylcholine and neostigmine increased the production amount of acetylcholine of the neuroblastoma, thereby causing the mimicking of parasympathetic nerve-induced stimulation. Further, the efficacy of the drug for the disease was evaluated by treating the neurological sialadenosis model with Botox.

### Example 4. Mouse Salivary Gland Organoid-Based Neurological Sialadenosis Model and Botox Potency Test Using The Same

4.1. Determination of Concentrations of Acetylcholine, Neostigmine, GT1b, and Botox that do not Affect Mouse Salivary Gland Organoids

Mouse salivary gland organoids were obtained using the same method as Experimental Example 3 above, and the composition of the culture medium employed during this experiment is as shown in Table 4 below.

TABLE 4

| Advanced DMEM/F12 | Basal medium | |
|---|---|---|
| Glutamax | 1 | X |
| HEPES | 1 | X |
| P/S | 1 | X |
| Primocin | 1 | X |
| B27 | 1 | X |
| NAC | 1.25 | Mm |
| A83-01 | 0.5 | μM |
| mRSPO1 | 1% | |
| hNRG1 | 37.5 | ng/ml |
| mFGF1 | 79.15 | ng/ml |
| mFGF7 | 18.8 | ng/ml |
| Y-27632 | 10 | μM |

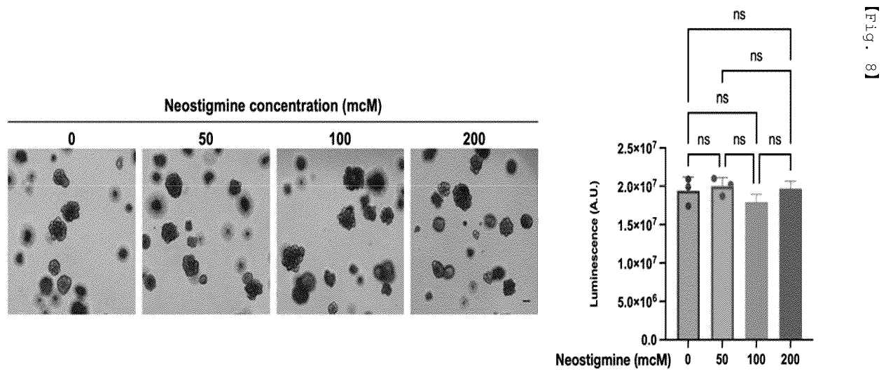

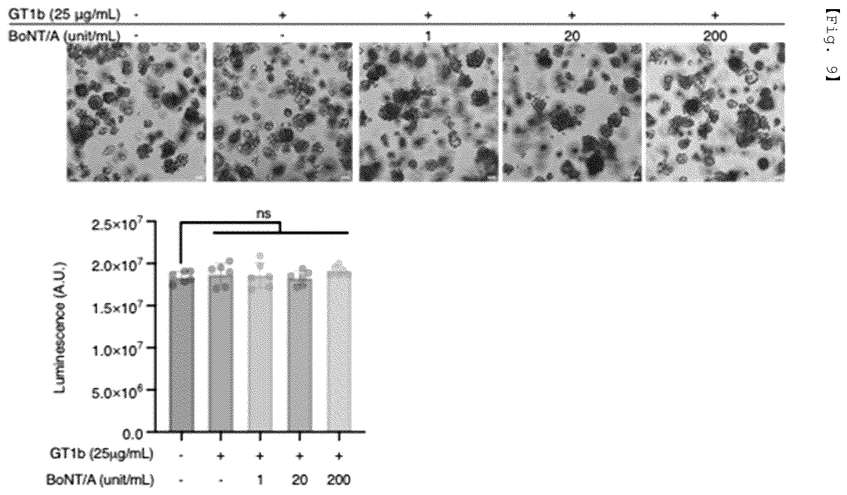

To prevent contamination of mesenchymal cells, etc., the effect of Botox on organoids was confirmed by observing cell morphology and measuring cell viability using mouse salivary gland organoids after passage 2 (FIG. 6). When various concentrations of acetylcholine were treated to mouse salivary gland organoids, it was confirmed that there was no effect on the morphology and growth of the organoids even when the concentration of acetylcholine was increased up to 100 nM (FIG. 7). Even when neostigmine was treated at 0, 50, 100, and 200 μM, no significant changes were observed in the morphology and growth of the organoids (FIG. 8). Further, it was confirmed that treatment with 25 μg/mL GT1b and various concentrations of Botox had no effect on the organoids (FIG. 9).

### 4.2. Co-Culture System of Mouse Salivary Gland Organoids and N2a Cells

N2a cells were cultured and subcultured using the same method as in Experimental Example 4 above, and the composition of the culture medium employed during this experiment is as shown in Table 5 below.

TABLE 5

| EMEM | Basal medium |
|---|---|
| P/S | 1X |
| FBS | 10% |

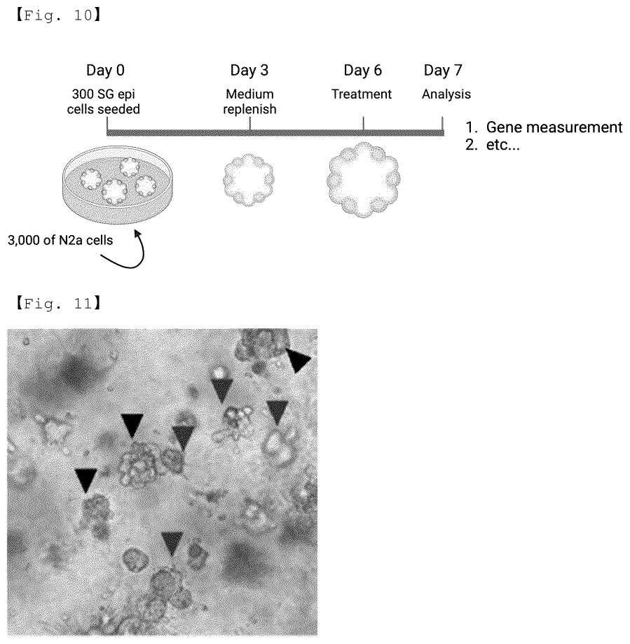

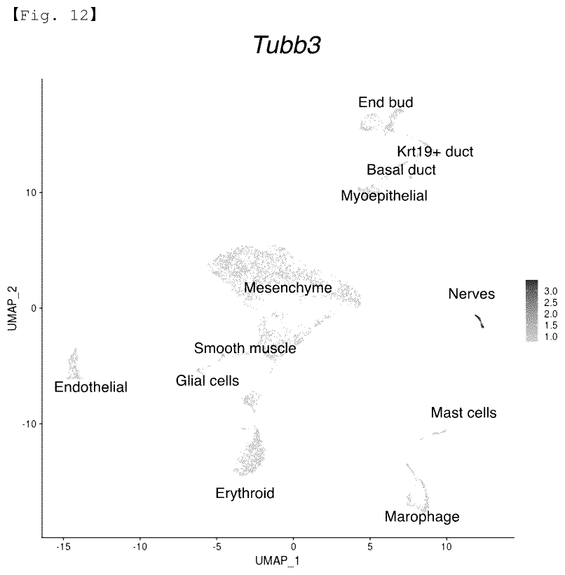

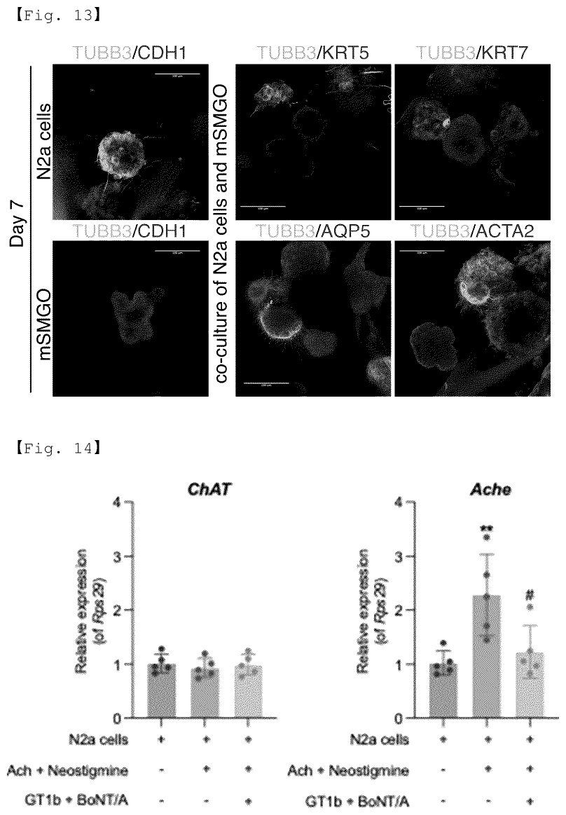

For co-culturing, 4 μL of organoid culture medium containing 300 cells forming mouse salivary gland organoids and 4 μL of organoid culture medium containing 3,000 N2a cells were mixed with 32 μL of Matrigel to form a 40 μL dome, followed by co-culturing by dispensing into a 24-well plate (FIG. 10). After 7 days of co-culture, the salivary gland organoids (black arrows) and the N2a cells (red arrows) were observed under a bright field microscope, as shown in FIG. 11. Single-cell transcriptome analysis results showed that the Tubb3 gene was not expressed in epithelial cells but was expressed in N2a cells, indicating that the gene is usable as a good indicator for distinguishing salivary gland organoids and N2a cells (FIG. 12). Based on this, immunofluorescence staining for KRT5 (basal cells), KRT7 (luminal cells), AQP5 (acinar cells), ACTA2 (myoepithelial cells), and TUBB3 confirmed that a co-culture system of salivary gland organoids and N2a cells was established (FIG. 13).

### 4.3. Botox Potency Test Using Co-Culture System of Mouse Salivary Gland Organoids and N2a Cells

On Day 6 of co-culture of salivary gland organoids and N2a cells, the cells were divided into three groups: Untreated (−Stim-Botox), Stimulated (+Stim-Botox), and Stimulated+Botox (+Stim+Botox) for the experiment. Co-treatment with acetylcholine 1 nM and neostigmine 200 μM was labeled +Stim, and co-treatment with GT1b 25 μg/mL and Botox 200 U/mL was labeled +Botox.

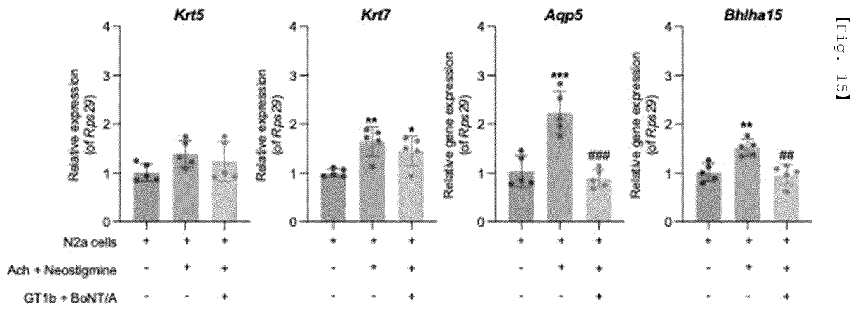

After 24 hours, the co-cultured wells were lysed directly with TRIzol, and mRNA was reverse transcribed into CDNA for qPCR. The ChAT gene, required for the synthesis of acetylcholine, which is predominantly expressed in N2a cells, did not change in the expression level even after 24 hours of Botox treatment, whereas the AchE gene, required for the degradation of acetylcholine, was increased by the treatment with acetylcholine and neostigmine (+Stim) and decreased again by Botox treatment (+Botox) (FIG. 14).

Furthermore, Krt5 (basal cells), Krt7 (luminal cells), Aqp5 (acinar cells), and Bhlha15 (acinar cells) expressed in salivary gland organoids, related to lumen or acinar cells, were increased by the treatment with acetylcholine and neostigmine and decreased again by Botox treatment (+Botox), with a similar trend observed in Bhlha15 (FIG. 15).

1.-24. (canceled)

25. A method for preparing a co-culture of a salivary gland organoid and a neuroblastoma, comprising:
(a) culturing a salivary gland organoid from a salivary gland tissue-derived epithelial cell in a culture medium containing a transforming growth factor-beta inhibitor (TGF-β inhibitor), a Wnt activator, a bone morphogenetic protein (BMP) inhibitor, a fibroblast growth factor (FGF) family, a receptor tyrosine kinase ligand, and a Rho-associated protein kinase (ROCK) inhibitor;
(b) culturing a neuroblastoma; and
(c) preparing a co-culture by mixing and culturing the salivary gland organoid and the neuroblastoma in a culture medium containing a TGF-β inhibitor, a Wnt activator, a BMP inhibitor, an FGF family, a receptor tyrosine kinase ligand, a ROCK inhibitor, and a cyclic adenosine monophosphate (cAMP) activator.

26. The method of claim 25, wherein the TGF-β inhibitor is any one selected from the group consisting of A83-01, SB-431542, SB-505124, SB-525334, SD-208, LY-36494, and SJN-2511.

27. The method of claim 25, wherein the Wnt activator is any one selected from the group consisting of R-spondin 1, R-spondin 2, R-spondin 3, and R-spondin 4.

28. The method of claim 25, wherein the BMP inhibitor is any one selected from the group consisting of Noggin, Dorsomorphin, DMH1, and LDN-193189.

29. The method of claim 25, wherein the FGF family is at least any one selected from the group consisting of FGF1, FGF2, FGF3, FGF4, FGF5, FGF6, FGF7, FGF8, FGF9 and FGF10.

30. The method of claim 25, wherein the receptor tyrosine kinase ligand is any one selected from the group consisting of Neuregulin β1 (NRG1), Heregulin β1 (HRG1), epidermal growth factor (EGF), transforming growth factor-α (TGF-α), basic fibroblast growth factor (bFGF), brain-derived neurotrophic factor (BDNF), hepatocyte growth factor (HGF), and keratinocyte growth factor (KGF).

31. The method of claim 25, wherein the ROCK inhibitor is any one selected from the group consisting of Y-27632, Fasudil, and H-1152.

32. The method of claim 25, wherein the CAMP activator is any one selected from the group consisting of Forskolin, Aminophylline, Pentoxifylline, Theophylline, Isobutyl-methylxanthine (IBMX), and Dehydroabietic acid (DAA).

33. The method of claim 25, wherein the culturing of the salivary gland organoid from the salivary gland tissue-derived epithelial cell is performed in a culture medium consisting of A83-01, R-spondin 3, Noggin, FGF2, FGF10, Neuregulin β1 (NRG1), and Y-27632.

34. The method of claim 25, wherein the preparing of the co-culture by mixing and culturing the salivary gland organoid and the neuroblastoma is performed in a culture medium containing A83-01, R-spondin 3, Noggin, FGF2, FGF10, Neuregulin β1 (NRG1), Y-27632, and Forskolin.

35. The method of claim 25, wherein the culture medium further contains one or more components selected from Glutamax, HEPES, Primocin, Prostaglandin E2 (PGE2), N-acetylcysteine (NAC), B27, and Nicotinamide.

36. A method for preparing a neurological sialadenosis model, comprising:
(a) culturing a salivary gland organoid from a salivary gland tissue-derived epithelial cell in a culture medium containing a transforming growth factor-beta inhibitor (TGF-β inhibitor), a Wnt activator, a bone morphogenetic protein (BMP) inhibitor, a fibroblast growth factor (FGF) family, a receptor tyrosine kinase ligand, and a Rho-associated protein kinase (ROCK) inhibitor;
(b) culturing a neuroblastoma;
(c) preparing a co-culture by mixing and culturing the salivary gland organoid and the neuroblastoma in a culture medium containing a TGF-β inhibitor, a Wnt activator, a BMP inhibitor, an FGF family, a receptor tyrosine kinase ligand, a ROCK inhibitor, and a cyclic adenosine monophosphate (cAMP) activator; and
(d) treating the co-culture with a neurotransmitter.

37. The method of claim 36, wherein the culturing of the salivary gland organoid from the salivary gland tissue-derived epithelial cell is performed in a culture medium containing A83-01, R-spondin 3, Noggin, FGF2, FGF10, Neuregulin β1 (NRG1), and Y-27632.

38. The method of claim 36, wherein the preparing of the co-culture by mixing and culturing the salivary gland organoid and the neuroblastoma is performed in a culture medium containing A83-01, R-spondin 3, Noggin, FGF2, FGF10, Neuregulin β1 (NRG1), Y-27632, and Forskolin.

39. The method of claim 36, wherein the neurotransmitter is any one selected from the group consisting of acetylcholine, dopamine, norepinephrine, serotonin, and histamine.

40. The method of claim 39, wherein the neurotransmitter is acetylcholine.

41. A method for confirming responses to a botulinum toxin drug in a neurological sialadenosis model, comprising: treating the neurological sialadenosis model prepared according to claim 36 with a botulinum toxin drug.

42. The method of claim 41, further comprising: confirming that an expression level of at least any one selected from the group consisting of KRT7, KRT19, AchE, and Aqp5 is reduced in the neurological sialadenosis model treated with the botulinum toxin drug compared to the control group not treated with the botulinum toxin drug.

43. The method of claim 41, wherein the botulinum toxin drug is Botox®, Meditoxin, Dysport®, Botulax, Myobloc®, BTX, or Xeomin®.

44. The method of claim 43, wherein the botulinum toxin drug is Botox®.
